(19) United States
(12) Patent Application Publication
(10) Pub. No.: US 2020/0345280 A1
(43) Pub. Date: Nov. 5, 2020

(54) SYSTEM AND METHOD FOR MONITORING AND CONTROLLING A PHYSIOLOGICAL CONDITION

(71) Applicant: United Arab Emirates University, Al Ain (AE)

(72) Inventors: Basma Nasser AlShawoosh, Al Ain (AE); Mousa Hussein, Al Ain (AE)

(21) Appl. No.: 16/398,886

(22) Filed: Apr. 30, 2019

(51) Int. Cl. A61B 5/145; A61B 5/00; G01R 33/20

(52) U.S. Cl. CPC A61B 5/14532; A61B 5/681; A61B 5/6822; A61B 5/0004; G01R 33/20

(57) ABSTRACT

There is provided a device, system and method for continuously monitoring a physiological condition such as diabetes in a body. The disclosed method comprises detecting a variation in capacitance value of a sensor coil when the sensor coil is placed in vicinity of the body and displaying the detected variation on a display unit, wherein the sensor coil has a fixed inductance value. The display unit is further integrated with a communication unit for communicating a monitored physiological condition in the body with an external communicating unit. The proposed device is non-invasive and in a form of a wearable.

### FIELD OF THE INVENTION

The present invention relates to the field of monitoring a physiological condition, and more particularly to a non-invasive system and method for monitoring and controlling glucose levels in blood.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Physiological monitoring is the basis of evaluative or analytic healthcare. The monitoring allows for identifying the dynamics or instabilities in blood flow (hemodynamics) and thereby initiate therapy or access other countermeasures for combating the same. However, the utility of most hemodynamic monitoring remains unproven and rather serves as a trigger for detection of multiple instabilities. Accordingly, continuous monitoring is a valuable tool that helps provide additional information to medical and nursing staff about physiological conditions of a patient. Using this information, clinical staff can better evaluate a patient's condition and thereby make appropriate treatment decisions.

Controlling diabetes is of utmost significance and even a matter of life or death in recent times considering that millions around the world suffer from it, and millions die of it. An interview and survey conducted with more than 160 diabetes patients pointed out that that majority of the patients faced problems with monitoring and controlling the glucose levels in their blood. These problems included the device used for monitoring being painful considering that users need to prick their fingers each time, making the patients to purposely avoid checking their glucose levels. However, diabetic patients should mandatorily check the same regularly in order to avoid diseases. Further, the sensors and strips or lancets being employed within the monitoring device itself are expensive.

Diabetic patients must be aware of the relationship between carbohydrates, medications and glucose levels as this particular relationship is crucial in controlling glucose levels. Guardians are constantly worried about a sudden hypoglycemic or hyperglycemic situation which may be encountered by their children due to which blood glucose levels are checked approximately every other hour.

Accordingly, there exists a need to provide a system and method for safe and continuous monitoring and controlling of glucose levels in the blood.

### SUMMARY OF THE INVENTION

Therefore it is an object of the present invention to provide a non-invasive system and method for safe and continuous monitoring and controlling of a physiological condition in a body.

The present invention proposes a non-invasive device for continuously monitoring a physiological condition in a body, the device comprising a linear sensor coil wound on a non-magnetic core, wherein the linear sensor coil monitors a concentration level of a parameter in blood based on detecting a change in capacitance of the linear sensor coil when the sensor coil is placed in vicinity of the body.

In an embodiment of the present invention, the physiological condition is diabetes, and the monitored parameter in blood is a glucose level.

In another embodiment of the present invention, the linear sensor coil has a fixed self-inductance.

In another embodiment of the present invention, self-inductance of the coil depends on a length of the linear sensor coil, number of turns of the linear sensor coil, spacing between turns of the linear sensor coil and a material of the linear sensor coil.

In another embodiment of the present invention, the change in capacitance of the linear sensor coil leads to a change in the resonant frequency of the linear sensor coil.

In another embodiment of the present invention, said non-invasive device is wearable as a wristband, watch, necklace or fitness gear.

As another aspect of the present invention, a non-invasive method of continuously monitoring a physiological condition in a body, the non-invasive method comprising the steps of detecting a variation in capacitance value of a sensor coil when the sensor coil is placed in vicinity of the body; and displaying the detected variation on a display unit, wherein the sensor coil has a fixed inductance value.

In another embodiment of the present invention, the physiological condition is diabetes.

In another embodiment of the present invention, the variation in capacitance value of the sensor coil is based on concentration level of a blood-based parameter.

In another embodiment of the present invention, the blood-based parameter is a glucose level.

In another embodiment of the present invention, the inductance value of the sensor coil depends on a length of the sensor coil, number of turns of the sensor coil, spacing between turns of the sensor coil and a material of the sensor coil.

As another aspect of the present invention, a system for continuously monitoring a physiological condition in a body is proposed, the system comprising a sensor coil wound on a non-magnetic core for monitoring a parameter in blood, an amplitude and phase comparator for comparing an input signal and an output signal from the sensor coil thereby resulting in a measured reading of the monitored parameter, a display unit in electrical communication with the amplitude and phase comparator for displaying the measured reading of the monitored parameter and a microcontroller in electrical communication with the sensor coil, amplitude and phase comparator and the display unit for converting the measured reading of the monitored parameter from the amplitude and phase comparator into a parameter standard format and displaying the converted reading on the display unit wherein the sensor coil monitors the parameter in blood based on detecting a change in capacitance of the sensor coil when the sensor coil is placed in vicinity of the body.

In another embodiment of the present invention, the physiological condition is diabetes, and the monitored parameter in blood is a glucose level.

In another embodiment of the present invention, the sensor coil has a fixed self-inductance.

In another embodiment of the present invention, self-inductance of the coil depends on a length of the sensor coil, number of turns of the sensor coil, spacing between turns of the sensor coil and a material of the sensor coil.

In another embodiment of the present invention, the change in capacitance of the sensor coil leads to a change in the resonant frequency of the sensor coil.

In another embodiment of the present invention, the change in the resonance frequency is detected by measuring an amplitude and phase variation of an output voltage of the sensor coil or a current flowing through the sensor coil.

In another embodiment of the present invention, the system further comprises a power supply for generating and inputting oscillatory electric currents to an oscillator and a voltage gain amplifier (VGA) for amplifying a signal received from the oscillator resulting in an amplified signal, and feeding the amplified signal to the sensor coil.

In another embodiment of the present invention, the display unit is integrated with a communication unit for communicating a monitored physiological condition in the body with an external communicating unit.

In another embodiment of the present invention, the said system is in a form of a wearable.

### BRIEF DESCRIPTION OF THE DRAWINGS

The subject matter that is regarded as the invention is particularly pointed out and distinctly claimed in the claims at the conclusion of the specification. The foregoing and other aspects, features, and advantages of the invention are apparent from the following detailed description taken in conjunction with the accompanying drawings in which—

FIG. 1 (a) and FIG. 1 (b) shows illustrates capacitance and resistance produced by an inductor, in accordance with the present invention.

FIG. 2 shows a proposed structure of the sensor coil, in accordance with the present invention.

FIG. 3 illustrates functioning of an amplitude and phase comparator, in accordance with the present invention.

FIG. 4 is a block diagram of the sensor along with necessary electrical components, in accordance with the present invention.

### DETAILED DESCRIPTION OF THE INVENTION

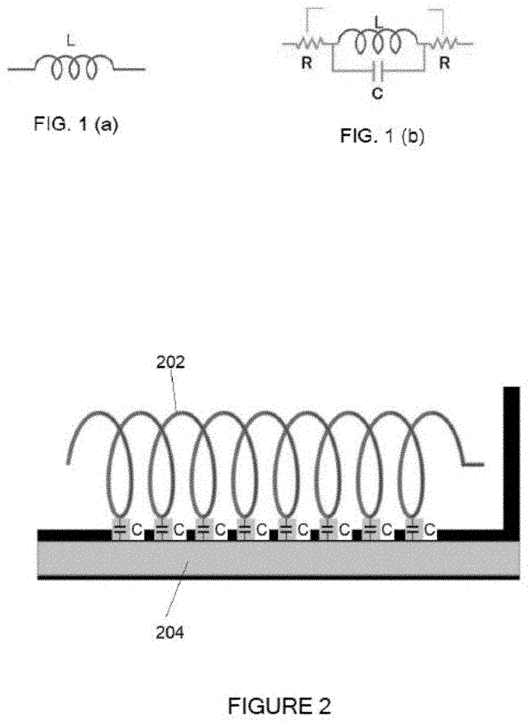

The aspects of the method or system to provide a nano memory system which allows to attain ultimate device down-scaling and increased charge retention capability according to the present invention, will be described in conjunction with FIGS. 1-4. In the Detailed Description, reference is made to the accompanying figures, which form a part hereof, and in which is shown by way of illustration specific embodiments in which the invention may be practiced. It is to be understood that other embodiments may be utilized and logical changes may be made without departing from the scope of the present invention. The following detailed description, therefore, is not to be taken in a limiting sense, and the scope of the present invention is defined by the appended claims.

The proposed system and method aims at designing and implementing a sensor for converting biological responses from a human body into electrical signals, and thereby sensing changes in the sugar content or glucose level within the human body. The sensor being employed is accurate, portable, safe and easy to use. Another advantage of the same is that the sensor is designed to be economically inexpensive, environmentally friendly and moreover does not cause any pain to the individuals using the sensor since the method is non-invasive. Considering the fact that continuous monitoring is a feature essential for the management of diabetic patients, the sensor in accordance with the present invention provides an advantage of ease of use. The sensor is placed on a body part such as the wrist or arm, wherein the sensor identifies glucose levels within the blood stream.

The device or sensor in accordance with the present invention comprises a simple linear coil wound on a non-magnetic core. The coil is an electrical element which experiences coil loss or winding loss, which is the energy dissipated by resistance in a wire used to wind a coil, and is represented as DC resistance R. This resistance R depends on the wire material, wire gauge, length of the wire, conductivity and ambient temperature. A capacitance exists in between a plurality of turns of the coil, due to a voltage gradient in the coil, which is basically the electric potential difference between any two points separated by a certain distance. Thereby, a difference in voltage creates an electrostatic field that causes a capacitive effect on the coil.

FIG. 1 illustrates capacitance and resistance produced by an inductor. The coil in accordance with the present invention, wound on a non-magnetic core results in self-inductance, which is defined as a property of the coil due to which it opposes the change of current flowing through it. Self-resonant frequency arises due to the presence of parasitic elements within an inductive circuit. Wire-wound inductors use a large amount of wire in the coils, and the wire itself has a parasitic resistance, which is in series with the ideal inductance. Also present is a parasitic capacitance in parallel with the series combination of the parasitic resistance and the ideal inductance, which arises because individual turns of the coil are in close proximity to one another. Accordingly, self-resonant frequency is expressed as:

f=1/2 π√LC

wherein f is the frequency, L is the inductance and C is the capacitance.

Accordingly, as the inductance L or capacitance C changes, this results in new resonant frequencies. Considering the design in accordance with the present invention, self-inductance of the coil is fixed, considering that it depends on a size of the wire, number of turns, spacing between the turns, and the core material used for the coil design. Hence, the present design is based on detecting a variation in capacitance values and not a variation in the inductance values.

In accordance with a preferable embodiment of the present invention, capacitance of the current system highly depends on a material present in the vicinity of the coil. Thereby, any change in the material under test (located near to the coil), leads to a change in the capacitance and accordingly, a change in the resonance frequency of the sensor. FIG. 2 shows a proposed structure of the coil 202 in accordance with the present invention. Accordingly, the proposed device or sensor consists of a flexible inductor, which comprises a linear coil 202 wound around a non-magnetic core. When the sensor or flexible inductor is brought into contact with a body 204, a new capacitance value is obtained, which is based on inductor stray capacitance between a plurality of turns of the coil.

The stray capacitance of inductors consists of three types of parasitic capacitances which are turn-to-turn, turn-to-layer, and turn-to-core capacitances. However, depending on a surrounding medium of the inductor or sensor coil 202, the stray capacitance may vary. In an embodiment of the present invention, one side of the sensor coil 202 is in contact with air and another side is in contact with the body 204, which has a different permittivity or dielectric constant depending on the body's glucose levels. Further, assuming that the body 204 will be the surrounding medium for a lower portion of the coil wires and that air will be the surrounding medium for the an upper portion of the coil wires, capacitance of the sensor coil 202 is expressed as:

C=εAd

wherein ε is the material permittivity or dielectric constant, A is the common area between coil turn and the surrounding material and d denotes a separation between turns of the coil and the material.

The sensor coil 202 in accordance with the present invention is fed with a sinusoidal input signal that passes through the sensor coil 202. In an embodiment, when no test material is present next to the sensor coil 202, the sensor resonates at a specific frequency, however, in the presence of a material in the vicinity of the sensor coil 202, the inductor (coil) stray capacitance changes. This capacitance change in turn will affect the resonance frequency of the sensor coil 202, and a new resonance frequency is obtained. There are several elements affecting the resonance frequency of the sensor coil structure, therefore a careful selection of the wire type, type of core, number and spacing of turns will affect the performance of the sensor coil 202.

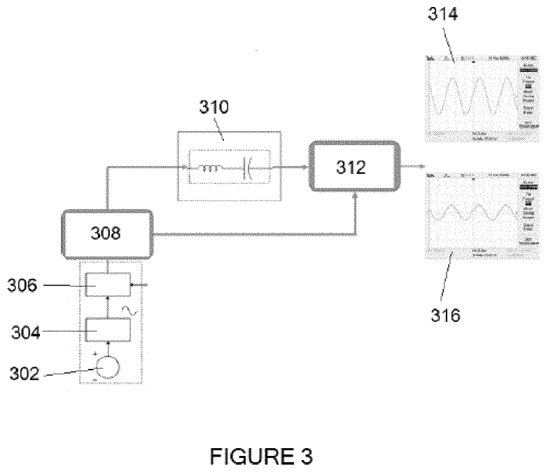

In an embodiment of the present invention, a change in the resonance frequency is easily detected by measuring an amplitude and phase variation of the sensor coil output voltage or a current flowing through the sensor coil. As the sensor contacts a body, it was observed that the resulting output waveform or signal becomes reduced compared to the case wherein the sensor is in contact with air. Therefore, the output waveform or signal leaving the sensor is fed to an amplitude and phase comparator, which compares the output waveform or signal with respect to the input waveform, as, denoted in FIG. 3. A DC power supply 302 of 5 to 12 V is fed to an oscillator 304 for generating oscillatory electric currents through a non-mechanical means. The generated signal is then fed to a voltage gain amplifier (VGA) 306 which is then forwarded to a signal generator 308. The generated signal from the signal generator 308 is fed to the sensor coil 310. In another embodiment, the signal generator 308 generates signals in the frequency range of 80 to 100 MHz.

The output signal leaving the sensor coil 310 is then fed to an amplitude and phase comparator 312. As denoted in FIG. 3, 314 shows the output waveform when the sensor coil is in contact with air and 316 shows the output waveform when the sensor coil is in contact with a body. The current proposed method is more accurate than traditionally practiced methods considering that a final reading is obtained using a method with two degrees of freedom, which is amplitude and phase variation. The glucose level detector or monitoring device measures a parameter based on signal amplitude variation and a frequency shift related to phase variations between an applied signal and an output of the sensor coil. In another embodiment of the present invention, the signal generator 308 and sensor coil 310 circuit along with the amplitude and phase comparator 312 is integrated with a display unit (not shown) and a Wi-Fi communications unit. The display unit is used to display the measurement readings in a glucose standard format, whereas the Wi-Fi communications unit relays the measured data to other units or programs such as a mobile application.

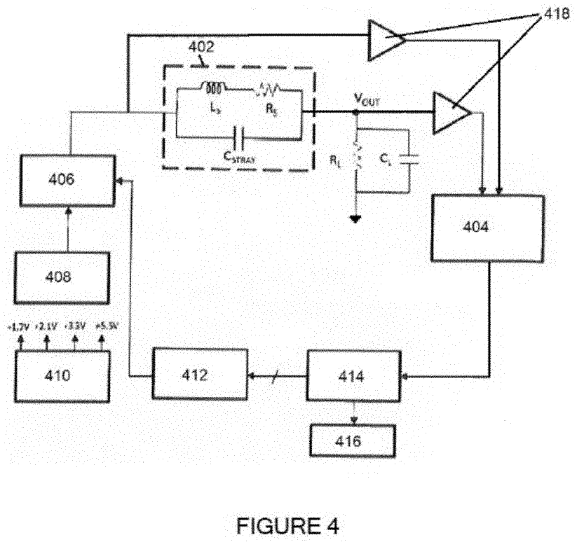

FIG. 4 is a block diagram depicting how each electrical component is integrated together in accordance with the present invention resulting in a complete and functional sensor. 402 is the currently proposed sensor, which shows a series connection of a stray resistance and ideal inductance of the sensor coil in parallel with stray capacitance. A DC power supply 410 supplies power to an oscillator 408 which produces a periodic oscillating electronic signal, to be fed to a variable gain amplifier (VGA) 406, an electronic amplifier that varies its gain (the ability to increase the power or amplitude of a signal from the input to the output port) depending on a control voltage. The oscillator 408 also converts DC current from the power supply 410 to an alternating current (AC) signal. An output signal from the variable gain amplifier (VGA) 406 is fed as input to the sensor 402. An output signal (Vₒᵤₜ) from the sensor 402 is passed on to an amplitude and phase detector 404, wherein the input signal fed to the sensor 402 is compared with this output signal (Vₒᵤₜ). A microcontroller 414 is connected to enable working of a display 416 for the system. 412 denotes a digital to analog converter unit and 418 denotes amplifiers used within the electrical circuit for amplifying signals fed to the same.

In another embodiment of the present invention, the results or final readings obtained from are analyzed to determine changes in glucose concentrations. The proposed device is used to continuously monitor glucose levels in the blood and subsequently displays the readings on a display screen. The display screen incorporates and alarm system for indicating a hypoglycemic or hyperglycemic condition. Such conditions may also be indicated as, but not limited to, an emoji or vibrational indications. On detection of severe situations, the current device or apparatus alerts a central emergency server or an emergency call center. Moreover, the system in accordance with the present invention has a capability to connect to mobile applications using Wi-Fi technology, wherein the particular mobile application provides a patient with additional health related information. Patients may simultaneously view analysis and statistics of their respective glucose levels. On the other hand, a patient may control their glucose levels by obtaining advice on preferable medications or dietary requirement plans based on a detected or measured level of glucose in the blood. In another embodiment, a patient may join diabetic forums or communities in order to exchange experiences, challenges and to encourage each other.

In another embodiment of the present invention, a non-invasive device is designed in the form of a wristband comprising a screen to continuously monitor glucose levels in the blood of a wearer and simultaneously displays the readings on the screen. Further, considering that the device is connected to an application, the wearer may view a real-time analysis and statistic of their respective glucose levels. As another application of the present wearable device, patients may measure a level of carbohydrates in food through capturing an image of the food and communicating or chatting with nutritionists in real-time. Also, considering diabetic patients who are children or elderly people, a guardian or caretaker may remotely monitor the patient and receive notifications or alerts on occurrence of a hypoglycemic or hyperglycemic condition. In the same way, paramedics or nurses may remotely monitor and obtain notifications regarding their respective patients. This particular way of controlling and monitoring will aid diabetic patients by avoiding any possible complications or life threatening occurrences, which may be caused by uncontrolled glucose levels in the blood. Accordingly, the verified feasibility of the system could play a major role in e-health.

Prominent advantages of the current invention include that the proposed system for glucose monitoring is easy, cost effective and non-invasive and may be used for continuously monitoring the glucose levels in blood without finger pricking or any such methods, which requires blood for conducting the test. Also, considering application of the present system as a wearable or watch, alarms or vibrational alerts enable rapid counter measures to be taken. The feature wherein the wearable is capable of communicating with an emergency center or hospital allows doctors to extract real-time reports, and to provide recommendations to patients regarding carbohydrate intake, required medications and exercises based on the received glucose level readings. In another embodiment, multiple wearable devices are synced using a single account thereby enabling ease of exchanging data and for communication. The present system or monitoring device helps to control and manage a diabetic life style avoiding complications and severe repercussions. Further, round the clock monitoring of a patient becomes possible considering that the device (in the form of a wearable) is worn by a patient at all times.

In another embodiment of the present invention, the glucose level monitoring device in accordance with the present invention provides a customized design for a wearer based on their gender (male or female) or age (children, teenagers, adults or senior citizens). Further, the proposed device is easy to use, cost effective, user friendly, compact, water resistant and presents a hassle-free way of monitoring glucose levels of a wearer. In another embodiment, the sensor coil device in accordance with the present invention is in a wearable form such as a wristband, watch, necklace or a fitness gear.

In another embodiment of the present invention, the proposed device is used for monitoring any physiological condition in a body wherein the sensor coil monitors a concentration level of a parameter in blood based on detecting a change in capacitance of the linear sensor coil when the sensor coil is placed in vicinity of the body. The monitored physiological condition includes, but is not limited to any one of, diabetes, thalassemia or anemia.

Many changes, modifications, variations and other uses and applications of the subject invention will become apparent to those skilled in the art after considering this specification and the accompanying drawings, which disclose the preferred embodiments thereof. All such changes, modifications, variations and other uses and applications, which do not depart from the spirit and scope of the invention, are deemed to be covered by the invention, which is to be limited only by the claims which follow.

1. A non-invasive device for continuously monitoring a physiological condition in a body, the device comprising:
a linear sensor coil wound on a non-magnetic core, wherein the linear sensor coil is configured to receive an input signal which travels through the linear sensor coil and is emitted as an output signal,
an amplitude and phase comparator configured to compare a variation of the output signal to the input signal, wherein a microcontroller uses an output from the amplitude and phase comparator along with the input signal generated using a signal generator to determine a change in resonance frequency, and
a display unit for displaying a final reading, wherein the final reading is obtained by converting the determined change in resonance frequency into a glucose standard format using the microcontroller,
wherein the final reading is based on signal amplitude variation and a frequency shift related to phase variations between the input signal and the output signal of the linear sensor coil, and
wherein resonance frequency of the linear sensor coil is initially measured for calibration without placing a test material next to the linear sensor coil.

2. The non-invasive device of claim 1, wherein the physiological condition is diabetes, and glucose level in blood is monitored.

3. The non-invasive device of claim 1, wherein the linear sensor coil has a fixed self-inductance.

4. The non-invasive device of claim 3, wherein self-inductance of the coil depends on a length of the linear sensor coil, number of turns of the linear sensor coil, spacing between turns of the linear sensor coil and a material of the linear sensor coil.

5. The non-invasive device of claim 1, wherein a change in capacitance of the linear sensor coil leads to a change in the resonant frequency of the linear sensor coil.

6. The non-invasive device of claim 1, wherein said non-invasive device is wearable as a wristband, watch, necklace or fitness gear.

7. A non-invasive method of continuously monitoring a physiological condition in a body, the non-invasive method comprising the steps of:
receiving an input signal which travels through a linear sensor coil and is emitted as an output signal, wherein the linear sensor coil is wound on a non-magnetic core;
measuring an amplitude and phase variation of the output signal from the linear sensor coil;
wherein a microcontroller uses the measured amplitude and phase variation of the output signal along with the input signal generated using a signal generator for determining a change in resonance frequency; and
displaying a final reading using a display unit, wherein the final reading is obtained by converting the determined change in resonance frequency into a glucose standard format using the microcontroller, wherein the final reading is based on signal amplitude variation and a frequency shift related to phase variations between the input signal and the output signal of the linear sensor coil, and
wherein resonance frequency of the linear sensor coil is initially measured for calibration without placing a test material next to the linear sensor coil.

8. The non-invasive method of claim 7, wherein the physiological condition is diabetes.

9. The non-invasive method of claim 7, wherein capacitance value of the linear sensor coil is based on concentration level of a blood-based parameter.

10. The non-invasive method of claim 9, wherein the blood-based parameter is a glucose level.

11. The non-invasive method of claim 7, wherein inductance value of the linear sensor coil depends on a length of the linear sensor coil, number of turns of the linear sensor coil, spacing between turns of the linear sensor coil and a material of the linear sensor coil.

12. A system for continuously monitoring a physiological condition in a body, the system comprising:
a sensor coil wound on a non-magnetic core for monitoring a parameter in blood and configured to receive an input signal which travels through the sensor coil and is emitted as an output signal,
an amplitude and phase comparator configured to compare a variation of the output signal to the input signal,
a microcontroller for determining a change in resonance frequency using an output from the amplitude and phase comparator along with the input signal generated using a signal generator,
a display unit for displaying a final reading, wherein the final reading is obtained by converting the determined change in resonance frequency into a glucose standard format,
wherein the microcontroller is in electrical communication with the sensor coil, amplitude and phase comparator and the display unit for converting the determined change in resonance frequency into a glucose standard format and for displaying the final reading on the display unit, wherein the change in the resonance the final reading is based on signal amplitude variation and a frequency shift related to phase variations between the input signal and the output signal of the linear sensor coil, and
wherein resonance frequency of the linear sensor coil is initially measured for calibration without placing a test material next to the linear sensor coil.

13. The system of claim 12, wherein the physiological condition is diabetes, and the monitored parameter in blood is a glucose level.

14. The system of claim 12, wherein the sensor coil has a fixed self-inductance.

15. The system of claim 14, wherein self-inductance of the coil depends on a length of the sensor coil, number of turns of the sensor coil, spacing between turns of the sensor coil and a material of the sensor coil.

16. The system of claim 12, wherein a change in capacitance of the sensor coil leads to a change in the resonance frequency of the sensor coil, since capacitance is directly proportional to resonance frequency.

17. The system of claim 16, wherein the change in the resonance frequency of the sensor coil is detected by measuring current flowing through the sensor coil.

18. The system of claim 12, the system further comprising:
a power supply for generating and inputting oscillatory electric currents to an oscillator; and
a voltage gain amplifier (VGA) for amplifying a signal received from the oscillator resulting in an amplified signal, and feeding the amplified signal to the sensor coil.

19. The system of claim 12, wherein the display unit is integrated with a communication unit for communicating a monitored physiological condition in the body with an external communicating unit.

20. The system of claim 12, wherein said system is in a form of a wearable.
